(19) United States
(12) Patent Application Publication
(10) Pub. No.: US 2013/0345514 A1
(43) Pub. Date: Dec. 26, 2013

(54) PROPRIOCEPTIVE ENDOSCOPE AND VIRTUAL DYNAMIC TOMOGRAPHY

(71) Applicant: Michael Keoni Manion, New South Wales (AU)

(72) Inventor: Michael Keoni Manion, New South Wales (AU)

(73) Assignee: Empire Technology Development LLC, Wilmington, DE (US)

(21) Appl. No.: 13/988,010

(22) PCT Filed: Jun. 22, 2012
(86) PCT No.: PCT/US12/43704

§ 371 Date: May 16, 2013

(51) Int. Cl. A61B 1/008

(52) U.S. Cl. CPC A61B 1/008
USPC 600/117; 600/142

(57) ABSTRACT

Some embodiments provided herein relate to a device for endoscopy. The device includes a first segment of endoscopic tube, a second segment of endoscopic tube, and at least one positional sensor configured to determine a relative orientation of the first segment to the second segment. Some embodiments provided herein relate to mapping information (such as images) along a specific path and/or length of an endoscopic probe.

### FIELD

The present application relates generally to the field of endoscopy.

### BACKGROUND

Endoscopy generally refers to a procedure that allows a physician to look inside the body of a patient using an endoscope. Endoscopes can have small cameras attached to long, thin tubes. The physician can moved the endoscope through a body opening, such as the mouth, to inspect an internal area of the body, such as the GI tract.

### SUMMARY

In some embodiments, an endoscopic device is provided. The device includes a first segment of endoscopic tube, a second segment of endoscopic tube, and a positional sensor configured to determine relative orientation of the first segment to the second segment.

In some embodiments, a method of endoscopy is provided. The method includes determining a first orientation of a first part of a tube of an endoscope, determining a second orientation of a second part of the tube of the endoscope, and combining the first orientation and the second orientation to provide a predicted configuration of the endoscope tube during an endoscopic procedure.

In some embodiments, a method of endoscopy is provided. The method includes providing a representation of a configuration of at least a part of a tube of an endoscope, providing one or more images of a location, and using the representation to modify the one or more images.

In some embodiments, a kit for endoscopy is provided. The kit includes an endoscopic tube and a measuring device. The endoscopic tube includes a first segment, a second segment, and a positional sensor configured to determine a relative orientation of the first segment to at least the second segment. The measuring device is configured to measure at least one of a) a length of the endoscopic tube that passes through a reference point and/or b) a rotational change in the endoscopic tube.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIGS. 1A and 1B are drawings depicting an exemplary shift in the position of a region of interest before and/or during an endoscopic procedure.

FIG. 2 is a drawing depicting some embodiments of an endoscopic device.

FIGS. 3A-3C are drawings depicting some embodiments of a segment of an endoscopic device including positional sensors.

FIG. 4 is a drawing depicting some embodiments of a method of endoscopy.

FIG. 5 is a flow chart depicting some embodiments of a positional sensor.

FIG. 6 is a drawing depicting some embodiments of an endoscope.

FIG. 7 is a flow chart depicting an embodiment of a method of endoscopy.

FIG. 8A is a drawing depicting a representation of a slice of a 3-D image.

FIG. 8B is a drawing of a representation of a 3-D image disassembled into slices (as a series of 2-D images).

FIG. 8C is a drawing of a representation of reconfigured slices in vivo.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be used, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

Provided herein are devices and methods that can allow for the detection and/or mapping of the position of an endoscope (and thus the tissue around it) during use, and devices and methods for manipulating any pre-endoscopic process imagery (or other imagery or data), so as to be more representative of the location and/or position of the surrounding tissue during the endoscopic process.

Navigation during endoscopic procedures aids in the ability to determine orientation and positioning of the endoscope, which can help verify what the physician is viewing, measuring, or interacting with. Knowing the position and/or orientation of just the tip of an endoscope may not provide sufficient data with respect to orientation of the endoscope within the body. Endoscopic procedures in the GI tract, particularly, can cause significant movement of the soft tissue of the organs of the GI tract and the organs surrounding the GI tract. Furthermore, common complications, such as looping, bowing, and kinking, can result in misleading information about the location of the endoscope with respect to the gut, even if the tip position is known in 3D.

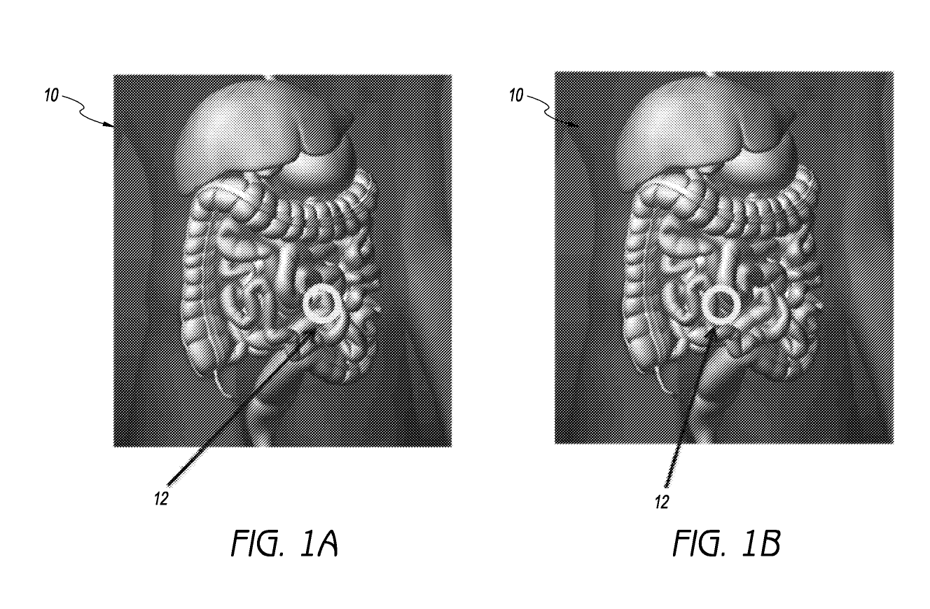

To some extent, endoscopic procedures can be enhanced by the availability of preoperative images obtained, for example, by MRI, CT, or PET scanning. These images can be useful for navigation during the endoscopic procedure, particularly if there is a region of interest (e.g., a tumor) to be examined. However, the position of the organs during the preoperative procedure can be quite different from the position of the organs during the endoscopic procedure, primarily due to the movement caused by the endoscope as it passes through the gut. For example, FIG. 1A shows an image 10 of the relative position of a region of interest 12 before endoscopy. In contrast, FIG. 1B shows how the region of interest 12 can shift during endoscopy. This issue is not merely one of convenience, for example, this shift can impact the sensitivity and specificity of EUS-FNA in non-small cell lung cancer staging. Furthermore, false negatives can be a difficult problem for EUS and EBUS, with some reports as high as 17% depending on lymph nodes biopsied. These inaccuracies can lead to misdiagnosis, or at best, to further, typically more invasive, procedures such as mediastinoscopy or thoracotomy with lymphadenectomy.

Being able to determine the shape of the gut or other tissue during an endoscopic procedure can allow for the adjustment of any useful preoperative imagery so as to correct the imagery for accurate use during the operation itself.

In some embodiments, an endoscopic device is provided. The device includes a first segment of endoscopic tube, a second segment of endoscopic tube, and at least one positional sensor configured to determine a relative orientation of the first segment to the second segment.

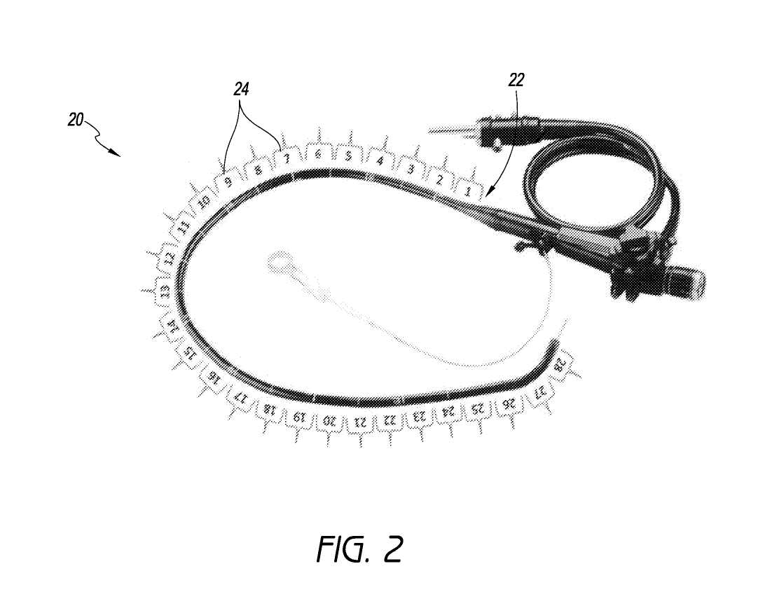

The device can include more than two segments. For example, the device can include about 2 to about 1000 segments. In some embodiments, there can be about one segment per centimeter of tube length. More segments are also possible. FIG. 2 depicts some embodiments of an endoscopic device 20 having a tube 22 that includes 28 different segments 24.

The first segment of the endoscopic tube can include a first positional sensor configured to determine an orientation and/or relative position of the first segment of the endoscopic tube to the second segment of endoscopic tube. In some embodiments, the first and/or second segments include more than one such positional sensor, such as two or three such sensors. As one progresses down the length of the tube, each segment can include one or more positional sensor configured to determine an orientation of that segment relative to the segment coming before it and/or following it. In some embodiments, all of the segments of the tube include one or a set of sensors. In some embodiments, only a subset of the segments includes one or a set of positional sensors.

In some embodiments, one or both of a first segment of endoscopic tube and a second segment of endoscopic tube includes at least one positional sensor configured to determine a length along one or more of an x-axis, a y-axis, and a z-axis of the segment. In some embodiments, the positional sensors can be stretch sensors (sensors capable of detecting a stretching motion) and the above can be achieved by employing three or more stretch sensors. The stretch sensors can measure the respective length of each segment along X, Y, and Z axes of the segment.

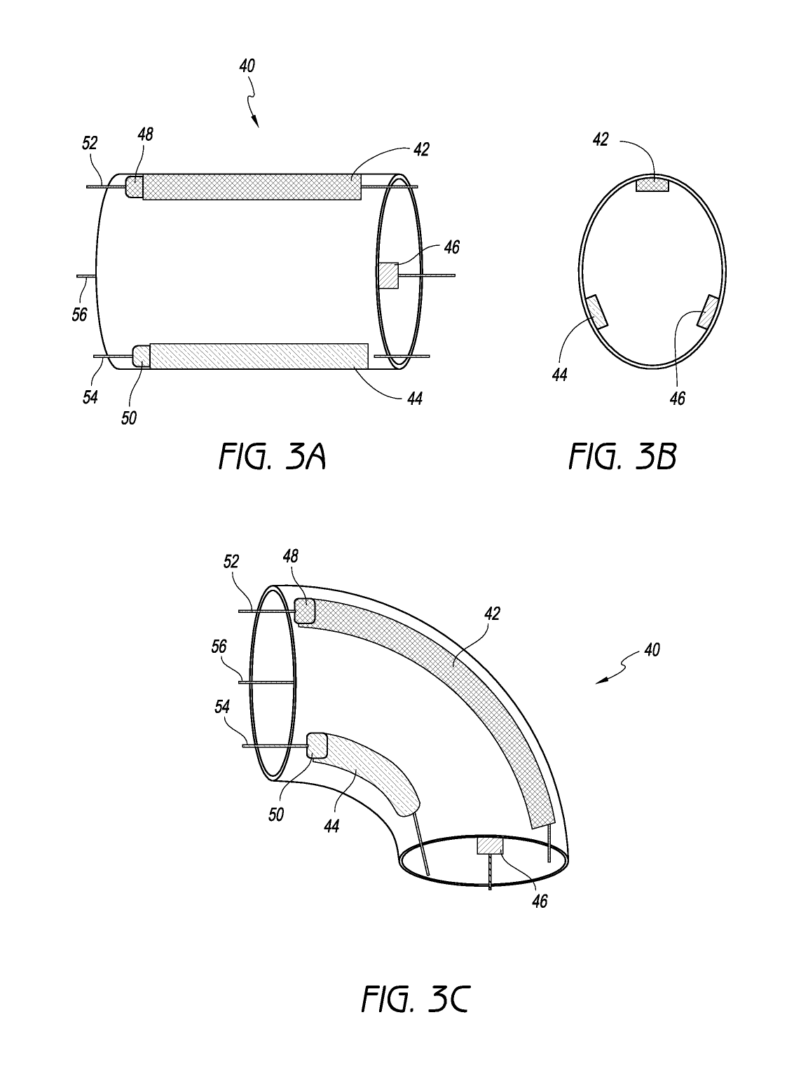

FIG. 3A depicts a segment 40 of an endoscopic device employing three positional sensors. The segment 40 includes an x-sensor 42, a y-sensor 46, and a z-sensor 44. FIG. 3B depicts an end view of the embodiment in FIG. 3A. FIG. 3C depicts the segment 40 being deformed, for example, when it is being used. As shown in FIG. 3C, the bend in segment 40 causes the x-sensor 42 to be stretched. In some embodiments, stretching the sensor can cause an increase in resistance, as the sensor will effectively become thinner, thereby increasing its resistance. In some embodiments, compressing the sensor can cause a decrease in resistance as the sensor will have an increase in diameter, and therefore have a lower resistance. Thus, by monitoring the electrical properties of sensors 42, 46, and 44, one can determine how much each has stretched or compressed. In some embodiments, nonelectrical measurements can be made as well, for example, time of light transmittance down segments of flexible fiber optics.

By mapping the stretching and/or compression out along the length of the tube, segment by segment, one can determine the stretch and/or compression at each segment, for each sensor, which can be mapped into a 3-D physical shape (or representation) for the endoscope.

In some embodiments, the sensors for each segment can be separate from the sensors upstream and/or downstream, and the stretch information can be transmitted down the length of the probe. In some embodiments, each sensor (or set thereof) can include input/output units configured to receive input from an upstream segment and transmit output to a downstream segment. For example, the x-sensor input/output unit 48 and the z-sensor input/output unit 50 are shown in FIG. 3A. The sensors 42, 46, 44 communicate through sensor connections 52, 56, 54.

In some embodiments, the positional sensors can integrate flexible electronics to receive data from an upstream sensor, measure the length of the current sensor, add that to the dataset, and send the updated dataset to the subsequent downstream sensor. For example, in the device of FIG. 2, Segment 10 can receive the x-lengths from x-sensors of segments 11-28, and add to the length the x-length for Segment 10, and provide this information to the x-sensor of Segment 9. The Y and Z sensors can work in the same way. The data can be used to reconstruct a 3D shape of an endoscope. The rate of data sampling can be rapid since the data is not very complex, allowing real time updating of the shape.

One or both of a first segment of endoscopic tube and a second segment of endoscopic tube can include more than one positional sensor. For example, in some embodiments, one or both of a first segment of endoscopic tube and a second segment of endoscopic tube includes two, three, four, five, six, or more positional sensors. There is no limitation on the number of sensors that can be present in each segment (that is, the number of sensors in a “set”); however, most applications will have adequate resolution with three (or even fewer) sensors along at least some of the segments.

In some embodiments, the positional sensor is positioned on an external surface of the endoscopic tube, which can provide for higher resolution. In some embodiments, the positional sensor is positioned inside of the first and second segments of the endoscopic tube (as shown in FIG. 3). For example, the sensor can be positioned within the tubing itself. The positional sensor can be configured to be attached to the device. The positional sensor(s) can be separate from the endoscope, and can be, for example, positioned on a sleeve that is configured to wrap around a section of the endoscopic tube. This can allow current endoscopic devices to use the positioning technology disclosed herein.

In some embodiments, the endoscopic tube and segments are flexible, and thus, the bending depicted in FIG. 3C will accurately depict the stretching of the sensors 42 and 46. In some embodiments, the endoscopic tube includes a series of rigid sections. In such embodiments, the stretching of the sensors can occur at the joints between each of the sections. Thus, the present embodiments are applicable to both flexible endoscopes and endoscopes that include rigid sections.

In some embodiments, the device can also include a torsion sensor between one or more of the segments. Such a sensor can be configured to detect torsional strains between two segments directly (rather than or in addition to the positional sensors). In some embodiments, this can be the same type of sensor as the positional sensor, but rather than being positioned to stretch and/or compress from flexing the segment, it can run along the circumference of the segments, at the joint between segments, such that it is attached at one end to the first segment and at the opposite end to the second segment, so that twisting between two segments results in the stretching and/or compression of the sensor. This is not required in all embodiments.

In some embodiments, the sensor is made from a conductive flexible material. In some embodiments, the positional sensor is made from a conductive elastomer. In some embodiments, the sensor can be made of conductive rubber and/or doped silicone.

In some embodiments, the sensor can be part of the actuators for controlling each segment of the endoscope. Thus, in some embodiments, the actuators for controlling the endoscope can include a positional sensor to allow the sensing of the stretching and/or compression. In some embodiments, the degree of movement and/or extent of actuation of the actuators in the tube can be used to determine a degree of deformation of each segment relative to another (or at least the tube as a whole). The device can monitor the actuation and thereby determine an approximation of the movement and/or reorientation of the various segments relative to one another.

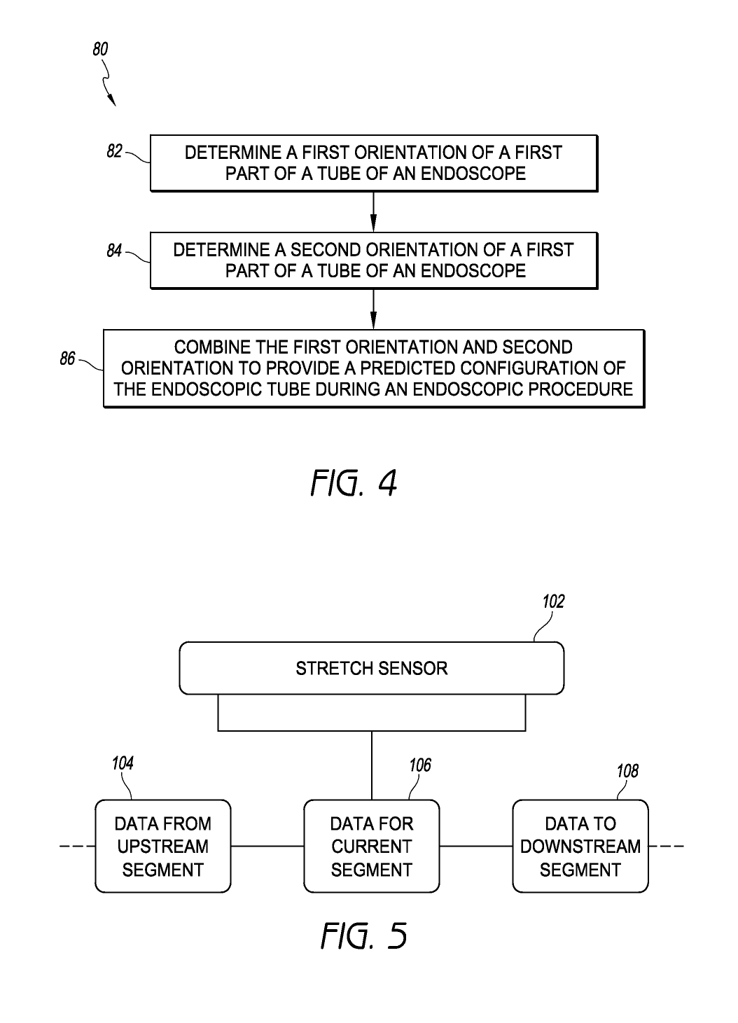

FIG. 4 depicts some embodiments of a method of endoscopy. The method 80 includes determining a first orientation of a first part of a tube of an endoscope 82, determining a second orientation of a second part of the tube of the endoscope 84, and combining the first orientation and the second orientation information to provide a predicted configuration of the endoscope tube during an endoscopic procedure 86. This configuration can be used to create a three-dimensional representation of the probe tube. In some embodiments, the first orientation is the relative position of a first segment of the tube to a second segment of the tube when the tube is in a resting arrangement (for example, the positional sensors are not deformed and/or are all equally deformed). In some embodiments, the second orientation is the relative position of the first segment of the tube to the second segment of the tube when the tube has been actuated (for example, at least one positional sensor is more stretched than the others in the same segment).

One or both of determining the first orientation and determining the second orientation is performed by one or more positional sensor (as outlined herein). For example, the sensors described with respect to FIGS. 3A-3C can be used to determine one or both of the first orientation and the second orientation. As noted above, each of the segments can have its relative position determined compared to the segment in front and/or behind it, via the one or more positional sensors (for example, three positional sensors, as depicted in FIG. 3). The positional sensor information can be used to determine the degree of deformation (via the extent of the stretching) and/or the direction of the deformation (via the combined information from the sensors in each segment (for example, the first, second and third sensor).

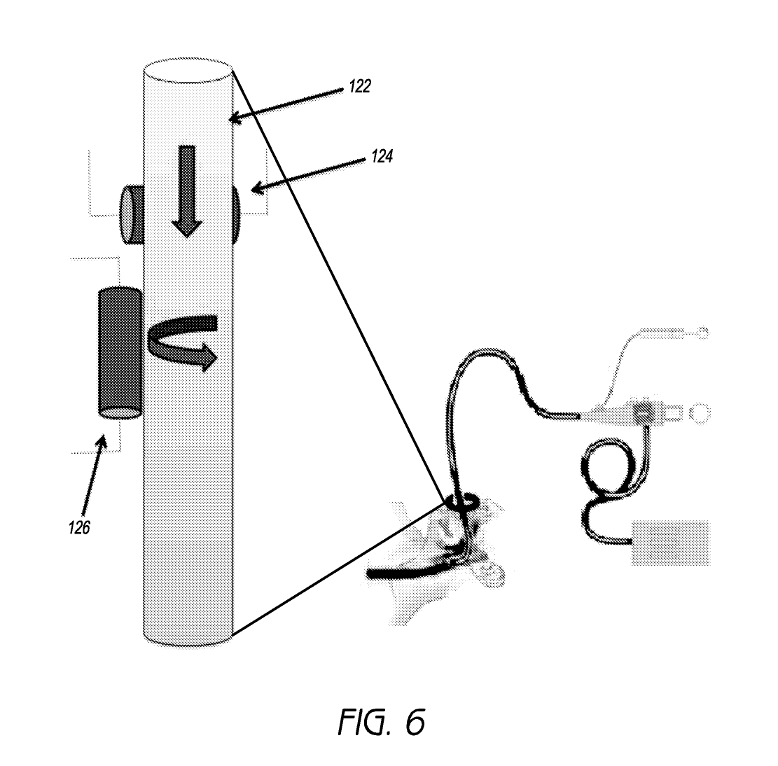

As noted above, the positional sensors can include integrated flexible electronics and can thereby receive data from an upstream sensor, measure the length of the current sensor, and add information that to the dataset. The dataset can then be sent to the subsequent downstream sensor. As shown in FIG. 5, the sensor 102 can receive data 104 from upstream segments, adds it to data 106 from the current segment and send the combined data 108 to the next downstream segment. In some embodiments, each sensor can be separately wired to an output from the probe, thus, integrated electronics need not be required in all embodiments. In some embodiments, each positional sensor, while in series with positional sensors upstream and/or downstream of the segment (electrically), can keep the stretch information discrete from other positional sensors by staggering the timing of the reporting of the positional sensor information. For example, the positional sensors from segment 1 of FIG. 2 can report resistance information the first millisecond of each second, segment 2 of FIG. 2 can report resistance information the tenth millisecond of each second, segment 3 of FIG. 2 can report resistance information the twentieth millisecond of each second, etc. Thus, by including a timing aspect on each sensor, one can also provide separate information of the activity of the sensor, along a single electrical path.

In some embodiments, additional and/or alternative embodiments regarding methods of endoscopy are provided. In some embodiments, the method of endoscopy includes determining a length of the tube of the endoscope that passes by a first reference point.

In some embodiments, the reference point can be at a site of insertion (for example, at the mouth). The length can be used to determine a distance of the end of the tube of the endoscope to the reference point. In some embodiments, the end of the tube includes an optical sensor, such as a lens. In some embodiments, the lens can be used to measure the length of scope passing through the reference point. In some embodiments, an optical sensor can measure markings on a surface of the endoscope as it is inserted into a subject to determine the length aspect. In some embodiments, mechanical sensors are used to determine a length of scope that has passed through the reference point. For example, rollers can be used to measure the length of the distal tip from the reference point as the endoscope is extended or retracted. The length of tube of the endoscope that passes by the first reference point can be combined with the predicted configuration to provide a first predicted location of the tube of the endoscope.

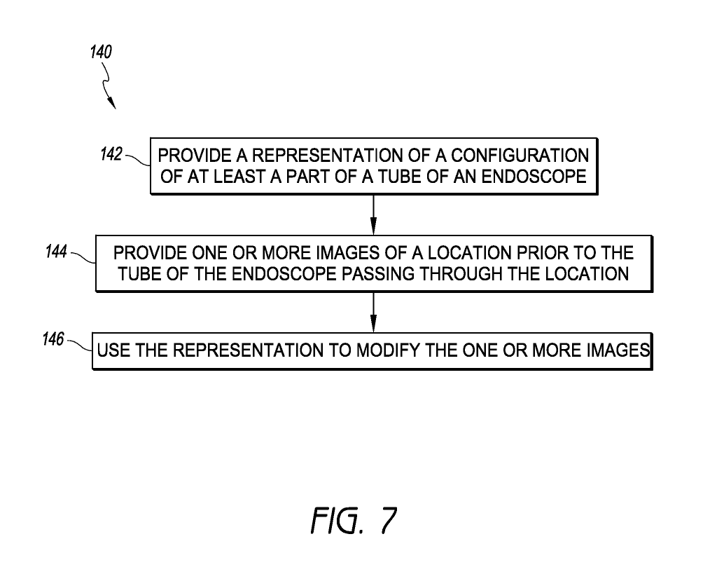

FIG. 6 depicts some embodiments of a device and/or system for determining the length of the tube of an endoscope that has been inserted into a subject. The device and/or system can include an endoscopic tube 122 and a roller 124. The roller can allow one to measure the length of insertion from a reference point as the scope is extended or withdrawn into a subject. In some embodiments, more than one roller can be employed so as to be able to measure both depth of insertion (how much of the length of the probe has passed a particular point) as well as rotational transformation. Thus, in some embodiments, the device can include a roller 126 which can be positioned so as to measure a rotational angle of the endoscope.

In some embodiments, a device is provided that includes one and/or two rollers. The first roller can be configured to provide a length measurement, as the length of the probe moves across the roller (where rotation of the roller corresponds to a length of the tube that has rolled over it). The second roller can be configured to provide a rotational measurement, as the turning of the probe will result in a turning of the second roller. The rollers can be coated with a high friction surface, so as to provide increased correlation between movement of the probe and movement of the roller. In some embodiments, the two rollers are provided on a frame, such that a probe can pass through the frame, allowing contact with both rollers, and assisting in keeping the probe in contact with the rollers during use. In some embodiments, the frame is configured such that it positions the rollers above the subject when in use.

In some embodiments, the torsion sensor provided herein can also be employed in determining a position of some part of the probe. In some embodiments, the method presumes that there is no torsional rotation or twisting of the endoscope.

In some embodiments, the change in rotational angle and depth of insertion determination (for example, as shown in FIG. 6) can be combined with the predicted configuration determination (for example, FIG. 4) to provide a first predicted location of the tube of the endoscope. Thus, not only can the configuration of the device be determined in some embodiments, but when combined with the depth of insertion and/or rotational information, one can map out where in the body some part of the tube of the probe is.

In some embodiments, the length of tube of the endoscope that passes by a first reference point is combined with the predicted configuration of the tube to provide a first predicted location of the endoscope, as described above. The rotational angle of the tube can further be used to adjust the first predicted location of the tube of the endoscope to provide a second predicted location of the tube of the endoscope. For example, data from both rollers 124, 126 (FIG. 6) can be combined with data from the positional sensors to recreate the 3-D shape of the endoscope and to provide a predicted location of the tube of the endoscope. This data can be a real time measurement of the shape of the gut of the patient at that time, as the endoscope can mark the lumen of the GI tract. In some embodiments, one uses the information to determine where an end of the endoscope is. In some embodiments, this allows one to map an image taken from the end of the endoscope to a particular location (via the position of the probe). In some embodiments, one uses the information to map out the full length of the endoscope, throughout the system. In some embodiments, one uses the information to verify that a sample to be taken from a particular location is actually being taken from the correct location. In some embodiments, one can use the information to determine how far the end of the endoscope is from a target location, such as a structure to be imaged or a tissue to be sampled. In some embodiments, it is useful to provide the mapping of the endoscope on a model of a body, wherein the model can be dynamically remodeled to know the exact configuration of the gut, for example, at any given point in time. In some embodiments, as noted below, the information can also be used to reconfigure pre-endoscopic imagery, to register the current endoscopic image more accurately. In some embodiments, the information is recorded to a computer readable medium. In some embodiments, the information (either “live” or from the computer readable medium) can be displayed as an image, to designate where at least a part of the probe was and/or is.

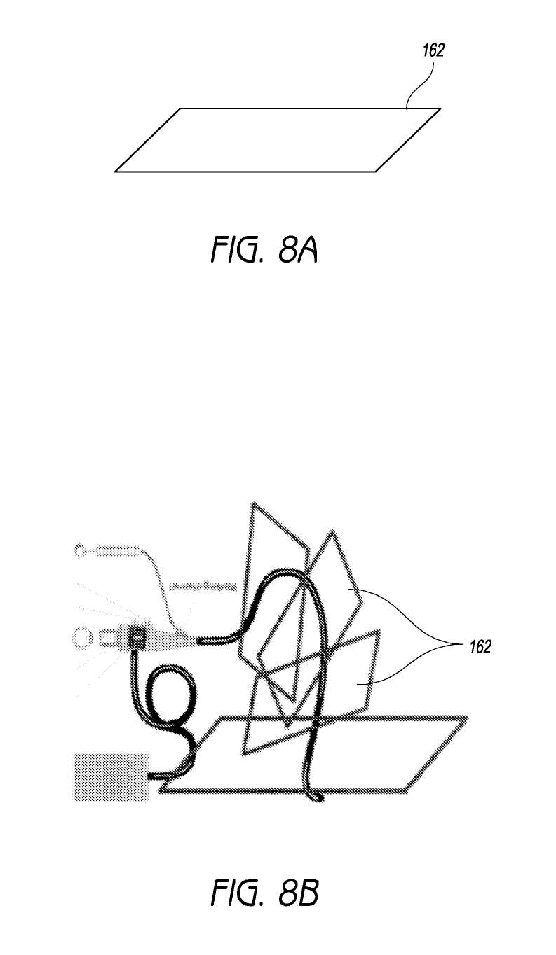

FIG. 7 depicts some embodiments of a method of endoscopy. The method 140 includes the providing a representation of a configuration of at least part of a tube of an endoscope 142, providing one or more images of a location prior to the tube of the endoscope passing through the location 144, and using the representation to modify the one or more images 146. The images can be taken of a location prior to the tube of the endoscope passing through the location. The images can be taken by any of a variety of imaging methods, for example endoscopy, PET, MRI, CT, or X-ray can be used.

Any of the embodiments provided herein, including any of their combinations, can be employed to remap the images that have been taken. In some embodiments, this can involve determining the predicted configuration of the endoscope tube (by using the sensors has provided herein). In such embodiments, the predicted configuration of the endoscope tube can be used as the representation of the configuration of at least part of the tube of the endoscope. For example, as described above, one or more positional sensors can be used to determine the relative location of one or more segments of an endoscope to provide a representation of a predicted configuration of a part of the endoscope. Then, the images can be remapped, for example in three-dimensional space, using the representation of the predicted configuration as a guide for positioning the tissue through which the probe passes. This can effectively redefine the location of the previous images, as the presence of the probe itself may have shifted the tissue around.

In some embodiments, the representation can be provided by determining a length of the tube of the endoscope that passes by a first reference point (e.g., using optical sensors, using mechanical sensors), as outlined above.

In some embodiments, the representation can be provided by determining a rotational angle of the tube of the endoscope relative to a second reference point, as described above.

In some embodiments, both the rotational angle of the tube of the endoscope and the length of the tube of the endoscope that passes by the first reference point can be combined with the predicted configuration of the endoscope tube to provide the representation of the configuration of at least a part of the tube of the endoscope. This can then be used for remapping of the images and/or other information. One can apply the representation to remap a model and/or update and/or adjust a prediction. The representation can also be used for adjusting non-medical imaging and/or plans, such as in topology and/or piping structures.

In some embodiments, the representation of the configuration of at least part of the tube of the endoscope represents a desired length of the probe, which can simply be a small part (such as the tip) or a longer length of the tube, for example the full length of the inserted tube. In some embodiments, the representation of the configuration of at least part of the tube of the endoscope represents at least 40% of a length of the tube of the endoscope that is within a subject. In some embodiments, the representation of the configuration of at least a part of the tube of the endoscope represents at least 80% of a length of the tube of the endoscope that is within a subject.

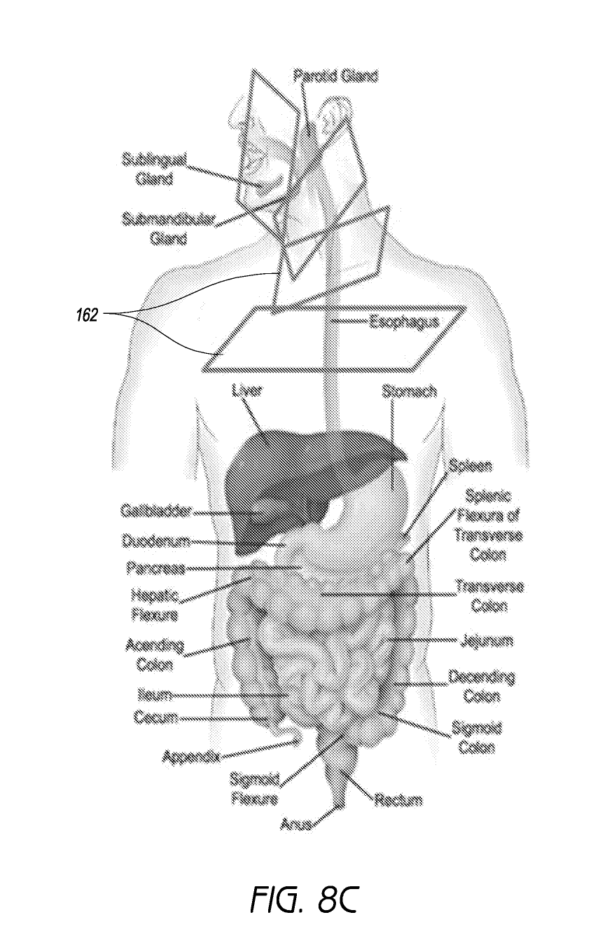

As noted above, in some embodiments, the one or more images is remapped and/or modified to more closely reflect a position of a structure in the image during the endoscopic procedure. The one or more images can be remapped or modified by treating each of the one or more images (or other piece of information associated with a pre-insertion environment) as a slice across a lumen of a canal in which the endoscope is inserted, and centering each slice using the representation to create a new orientation of the canal. A 3-D image can be modified by digitally disassembling the 3D image into individual slices (for example, tomogram slices) oriented horizontally across the lumen of the GI tract. FIG. 8A depicts an embodiment of a slice 162. In some embodiments, the center point of each slice is the middle of the lumen at that point along the GI tract. There may be exceptions for larger bodies such as the stomach, in which case, algorithms can provide guidance to orient the center of the slice with the most likely position of the endoscope within that space. FIG. 8B depicts an image of numerous slices 162 positioned along the length of the tube. FIG. 8C depicts the numerous slices 162, positioned in an exemplary manner over the lumen of the GI tract.

The representation of the configuration of at least a part of the tube of the endoscope can then be used to reposition the slices 162 by centering each slice 162 using the position of the endoscope corresponding to the area around each slice 162 as provided by the representation. The pre-endoscopic image can then be deconvolved using the new orientation to form a new image.

In some embodiments, only a certain depth surrounding the lumen of the tract being interrogated will be morphed to the new shape to reduce computing power required. With sufficient computing power, however, one can reorganize the entire internal structures based on information provided from the endoscope, using the body surface as the boundary. In some embodiments, the adjusted image and/or dataset, which takes the presence of the configuration of the endoscope into account, can be recorded to a computer readable medium and/or displayed to a user on a monitor or other screen.

In some embodiments, a kit for endoscopy is provided. In some embodiments, the kit includes any one or more of the embodiments provided herein.

In some embodiments, the kit includes an endoscopic tube including a first segment, a second segment, and at least one positional sensor. The positional sensor is configured to determine a relative orientation of the first segment to at least the second segment.

In some embodiments, the kit includes a measuring device configured to measure at least one of a) a length of the endoscopic tube that passes through a reference point or b) a rotational change in the endoscopic tube. The measuring device can include one or more of a mechanical sensor, an optical sensor, and a roller.

In some embodiments, the kit includes both of the above embodiments. In some embodiments, the kit includes one or more of the above embodiments, along with a computer readable medium that includes instructions and/or an algorithm for executing any of the methods provided herein. In some embodiments, the algorithm calculates the relative position of at least a first and second segment by determining the amount of resistance in three positional sensors positioned within an endoscopic probe or within a sleeve on the outside of an endoscopic probe and translating the resistance values into stretch values for determining the degree of bend between the segments. In some embodiments, the algorithm translates rotations of a roller into a linear displacement of a probe tube and/or a rotational displacement of the tube. In some embodiments, the algorithm collects a series of pre-endoscopic images and repositions them along a provided representation of the endoscopic tube.

As described above, in some embodiments, the devices, methods, and kits disclosed herein can reduce the risk for false negatives. False negatives can be a particularly troublesome problem. In some cases, they can lead to a misdiagnosis, which can result in severe consequences for the patient as they may not receive the necessary treatment. If the surgeon has a positive result on a pre-endoscopic test, and a negative result with EUS or EBUS (with or without FNA), they will have to decide whether to do a follow up procedure. Sometimes this is another EUS/EBUS, and sometimes this is a more invasive surgical biopsy. In both cases, however, this can add significant cost to the medical system, as well as risk and harm to the patient.

The devices, methods, and kits disclosed herein can include the endoscopic designs incorporating positional sensors described herein. Systems to retrofit existing endoscopic systems are provided. For example, a sleeve which endoscopes are inserted into can be provided, which can include the positional sensors. The sleeve can be disposable. As noted above, software and display systems can also be provided. For example, the software and display systems can be incorporated into existing systems, or new systems to allow for integrated display, navigation, and statistical guidance. In some embodiments, a software system can provide post-operative processing.

In some embodiments, the methods and devices provided herein allow for determining, predicting, and/or monitoring more than just the tip of the probe. In some embodiments, these allow for monitoring at least 50 percent of the length of the probe. In some embodiments, the 50% is the section that is closest to the distal end.

In some embodiments, the methods and/or devices allow for more than simply providing orientation and position just of the tip of the endoscope. In some embodiments, the device and/or method can provide information about the current shape of the GI tract (or other tract being interrogated).

In some embodiments, the device and/or method provides for more than 8 sensors containing segments for monitoring in real time. In some embodiments, the device and/or method provides sufficient resolution for EUS or EBUS applications in the small intestines or bronchial tubes. In some embodiments, the number and/or density of sensors is such that the 3-D shape and/or orientation of the endoscope along its entire length is provided. In some embodiments, utilizing the information from the shape of the endoscope to determine the shape of the gut during the procedure can allow a much more accurate registration of the disparate data source, both during the procedure and afterwards.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

In an illustrative embodiment, any of the operations, processes, etc. described herein can be implemented as computer-readable instructions stored on a computer-readable medium. The computer-readable instructions can be executed by a processor of a mobile unit, a network element, and/or any other computing device.

There is little distinction left between hardware and software implementations of aspects of systems; the use of hardware or software is generally (but not always, in that in certain contexts the choice between hardware and software can become significant) a design choice representing cost vs. efficiency tradeoffs. There are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; if flexibility is paramount, the implementer may opt for a mainly software implementation; or, yet again alternatively, the implementer may opt for some combination of hardware, software, and/or firmware.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a CD, a DVD, a digital tape, a computer memory, etc.; and a transmission type medium such as a digital and/or an analog communication medium (e.g., a fiber optic cable, a waveguide, a wired communications link, a wireless communication link, etc.).

Those skilled in the art will recognize that it is common within the art to describe devices and/or processes in the fashion set forth herein, and thereafter use engineering practices to integrate such described devices and/or processes into data processing systems. That is, at least a portion of the devices and/or processes described herein can be integrated into a data processing system via a reasonable amount of experimentation. Those having skill in the art will recognize that a typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems including feedback loops and control motors (e.g., feedback for sensing position and/or velocity; control motors for moving and/or adjusting components and/or quantities). A typical data processing system may be implemented utilizing any suitable commercially available components, such as those typically found in data computing/communication and/or network computing/communication systems.

The herein described subject matter sometimes illustrates different components contained within, or connected with, different other components. It is to be understood that such depicted architectures are merely examples, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable and/or physically interacting components and/or wirelessly interactable and/or wirelessly interacting components and/or logically interacting and/or logically interactable components.

### EXAMPLES

### Example 1

### Creating a Three-Dimensional Representation of an Endoscopic Probe

An endoscope as shown in FIG. 2 is provided. Each segment includes three positional sensors on the inside of the endoscope, space equally around the internal circumference of the probe tube. Each positional sensor measures an amount of stretch that occurs in that particular segment, along the length that the sensor runs. The degree of stretch is observed as a change in resistance of the specific sensor.

The tube of the endoscope is inserted into a subject and positioned proximally to a desired location. The shape of the endoscope tube is determined by using all three of the stretch sensor resistance values for each segment, to determine the relative position of each segment to the following segment, and each of these values is combined such that a full three dimensional representation of the probe is created.

### Example 2

### Identifying a Sample Area

The probe of Example 1 is inserted into the intestinal track of a subject to look for tumors in the subject. The probe is inserted and the surround tissue examined until a region of interest is identified in the subject. A tissue sample from a candidate tumor is taken by fine needle aspiration. The shape of the endoscope tube is determined by using all three of the stretch sensor resistance values for each segment, to determine the relative position of each segment to the following segment, and each of these values is combined such that a full three dimensional representation of the probe is created. The three-dimensional representation of the probe tube is recorded to a computer readable medium as a location within the subject that the particular sample was taken from. The location is further specified by using the representation, within the known organs that the probe tube has passed through, to denote more precisely wherein in the subject the sample was taken from.

### Example 3

### Locating a Desired Target Area

The sample taken from the subject in Example 2 is examined and determined to be cancerous. The subject is asked to come in again for removal of the cancerous region of interest.

The same type of endoscopic probe is inserted into the subject to a location proximal to the region of interest. This is done by monitoring both the tissue that the probe passes through, but also by monitoring the three-dimensional representation of the probe. The user will know that the end of the probe is at the desired target area when the three-dimensional representation of the probe tube closely matches the three-dimensional representation in Example 2.

### Example 4

### A Method of Repositioning CT Scanning Data During Endoscopy

A CT scan is used to produce 3-D images of the anatomy of the gut of a patient prior to an endoscopic procedure used to image a particular region of interest.

Endoscopy is performed within the GI tract to search for any abnormalities. Positional sensors along the endoscope and optical sensors at a proximal end of the endoscope are used to provide a representation of the endoscope in real-time during the procedure. The endoscopic process further includes rollers at the point of insertion of the endoscope that provide data reflecting the length of the tube of scope that passes by the point of insertion and reflecting the rotational angle of the tube of the endoscope relative to the point of insertion. The data reflecting the configuration of the endoscope tube, the length of the tube of scope passed by the point of insertion, and the rotational angle of the tube of the scope relative to the point of insertion are combined to provide a representation of the configuration of the endoscope.

The 3D image from the CT scan is deconvolved into individual slices centered along the lumen of the GI tract. The representation of the configuration of the endoscope is used to re-orient the slices and center them along the lumen of the endoscope as shown in the representation of its configuration, creating a new 3D image depicting the anatomy of the gut during endoscopy. The new 3D image can be used to aid in navigation of the endoscope to and from the region of interest.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

1. (canceled)

2. (canceled)

3. (canceled)

4. An endoscope device, the device comprising:
a first segment of endoscopic tube;
a second segment of endoscopic tube; and
a positional sensor configured to determine a relative orientation of the first segment to the second segment,
wherein one or both of the first segment of endoscopic tube and the second segment of endoscopic tube comprises a positional sensor configured to determine a length along one or more of an x-axis, a y-axis, and a z-axis of the segment.

5. (canceled)

6. (canceled)

7. (canceled)

8. (canceled)

9. (canceled)

10. (canceled)

11. A method of endoscopy, the method comprising:
determining a first orientation of a first part of a tube of an endoscope;
determining a second orientation of a second part of the tube of the endoscope;
combining the first orientation and the second orientation to provide a predicted configuration of the endoscope tube during an endoscopic procedure; and
determining a length of the tube of the endoscope that passes by a first reference point.

12. The method of claim 11, wherein the length is used to determine a distance of an end of the tube of the endoscope to the reference point.

13. The method of claim 12, wherein the end of the tube of the endoscope comprises a lens.

14. The method of claim 12, wherein the length of the tube of the endoscope that passes by a first reference point is combined with the predicted configuration to provide a first predicted location of the tube of the endoscope.

15. The method of claim 11, further comprising determining a change in a rotational angle of the tube of the endoscope relative to a second reference point.

16. The method of claim 15, wherein the first and the second reference points are a same reference point.

17. The method of claim 15, wherein the change in the rotational angle of the tube is used to adjust the first predicted location of the tube of the endoscope to provide a second predicted location of the tube of the endoscope.

18. (canceled)

19. (canceled)

20. (canceled)

21. (canceled)

22. (canceled)

23. (canceled)

24. A method of endoscopy, the method comprising:
providing a representation of a configuration of at least a part of a tube of an endoscope;
providing one or more images of a location, prior to the tube of the endoscope passing through the location; and
using the representation to modify the one or more images,
wherein the representation is provided by:
determining a first orientation of a first part of the tube of the endoscope;
determining a second orientation of a second part of the tube of the endoscope; and
combining the first orientation and the second orientation to provide a predicted configuration of the endoscope tube, wherein the predicted configuration of the endoscope tube is the representation of the configuration of at least a part of the tube of the endoscope, and
wherein the representation is provided by further determining a length of the tube of the endoscope that passes by a first reference point.

25. The method of claim 24, wherein the representation is provided by further determining a rotational angle of the tube of the endoscope relative to a second reference point.

26. The method of claim 25, wherein the rotational angle of the tube of the endoscope and the length of the tube of the endoscope that passes by the first reference point are combined with the predicted configuration to provide the representation of the configuration of at least a part of the tube of an endoscope.

27. (canceled)

28. (canceled)

29. (canceled)

30. (canceled)
